(19) United States
(12) Patent Application Publication
(10) Pub. No.: US 2016/0051678 A1
(43) Pub. Date: Feb. 25, 2016

(54) SUSPENSION PHARMACEUTICAL FORMULATIONS COMPRISING LOW MELTING PROPIONIC ACID DERIVATIVE PARTICLES

(71) Applicant: Johnson & Johnson Consumer Inc., Skillman, NJ (US)

(72) Inventors: Saumitra Bagchi, Princeton, NJ (US); Murali K. Vuppala, College, PA (US)

(21) Appl. No.: 14/837,738

(22) Filed: Aug. 27, 2015

Related U.S. Application Data
(62) Division of application No. 14/027,612, filed on Sep. 16, 2013.
(60) Provisional application No. 61/702,442, filed on Sep. 18, 2012.

(51) Int. Cl. A61K 47/14; A61K 31/192

(52) U.S. Cl. CPC A61K 47/14; A61K 31/192

(57) ABSTRACT

Low melting propionic acid derivative particles that are free flowing and have significantly reduced or eliminated throat burn are disclosed. A method of manufacturing the low melting propionic acid derivative particles; dosage forms containing the low melting propionic acid derivative particles; methods of manufacturing the dosage forms; and methods of treatment using the dosage forms are also disclosed.

This application claims priority of the benefits of the filing of U.S. Provisional Application Ser. No. 61/702,442, filed Sep. 18, 2012, the contents of each of which are hereby incorporated by reference in their entirety

### FIELD OF THE INVENTION

The present invention relates to low melting propionic acid derivative particles that are free flowing and have significantly reduced or eliminated throat burn or burning sensation in the mouth and throat. The invention also relates to methods of manufacturing the taste-masked low melting propionic acid derivative particles; methods of manufacturing controlled release low melting propionic acid derivative particles; dosage forms containing these low melting propionic acid derivative particles; methods of manufacturing the dosage forms; and methods of treatment using the dosage forms.

### BACKGROUND OF THE INVENTION

The present invention relates to low melting propionic acid derivative particles, and more specifically to low melting propionic acid derivative compositions containing low melting propionic acid derivative particles having reduced or no throat burn characteristics. The invention is particularly useful in the manufacture of dosage forms containing low melting propionic acid derivative compounds such as ibuprofen, ketoprofen, dexibuprofen, etc.

Administration of medicines to children is always a challenging task for caregivers mainly due to the bitter taste associated with many drugs. Chewable tablets or powders are one of many formulations that can overcome these challenges. Many flavors and sweeteners have been added to medication in order to make them more palatable and to mask the unpleasant taste and aftertaste which is common with many medications. Certain medicinal ingredients, in addition to having an unpleasant taste, create a burning or scratching sensation in the mouth and/or throat when administered as chewable tablets, swallowable powder/granules, suspensions and uncoated tablets. Flavors and sweeteners do little to overcome this throat burning sensation. Despite numerous efforts to find an effective means to eliminate this burn, there is a continuing need for a method to effectively eliminate the burning sensation with medications, preferably so that the burn can be reduced to a level such that a chewable composition can be provided.

Propionic acid derivatives are used to relieve pain, tenderness, swelling, and stiffness caused by osteoarthritis (arthritis caused by a breakdown of the lining of the joints) and rheumatoid arthritis (arthritis caused by swelling of the lining of the joints). They are also used to relieve mild to moderate pain, including menstrual pain (pain that happens before or during a menstrual period). Propionic acid derivatives are also used to reduce fever and to relieve mild pain from headaches, muscle aches, arthritis, menstrual periods, the common cold, toothaches, and backaches. For example, ibuprofen, a propionic acid derivative in a class of medications called NSAIDs, works by stopping the body's production of substances that cause pain, fever, and inflammation.

Propionic acid derivatives possess an unpalatable burning sensation in the mouth and throat after ingestion. Several approaches for overcoming this burning sensation have been proposed in the art.

Japanese Patent Application No. 91997-2949 to American Home Products attempts to eliminate the unpalatable aftertaste by providing only one enantiomer of ibuprofen. The application discloses the separation of ibuprofen from its racemic mixture to form an orally administered drug composition which contains only the S(+)-ibuprofen and essentially no R(−)-ibuprofen. While this approach may provide a more palatable form of ibuprofen, separation and isolation of enantiomers is difficult.

U.S. Pat. No. 5,320,855 to McNeil-PPC, Inc. discloses a method of masking the taste of ibuprofen by granulating with polyvinylpyrrolidone, sodium starch glycolate and sodium lauryl sulfate and coating the resulting granules with hydroxyethyl cellulose or a mixture of hydroxyethyl cellulose and hydroxypropyl methylcellulose. While resulting in a taste improvement, this method does not completely eliminate the “throat burn” associated with ibuprofen in chewable dosage forms.

U.S. Pat. Nos. 6,627,214 and 7,078,053 to McNeil-PPC, Inc. disclose a method for inhibiting the burn sensation of racemic mixtures of propionic acid derivatives by generally providing fumaric acid in an amount, relative to the propionic acid derivative dosage, of about 50 to about 150 weight percent. While fumaric acid can be effective at lowering the burn sensation, proportionally higher levels of fumaric acid may contribute to a level of sourness, which could render convenience dosage forms such as fast dissolving and chewable tablets less palatable. Another approach is to coat the ibuprofen particles with a hydro-colloid and fumaric acid in order to minimize the irritation to the mucous membranes of the throat as disclosed in U.S. Pat. No. 4,762,702 to Gergely et al. Because of their hydrophilicity, hydro-colloids permit water to be quickly absorbed into the drug particle upon ingestion, which disadvantageously reduces the burn masking effect of the coating. Yet a further approach is to mix an acid compound, such as fumaric acid, with an active ingredient coated with a tastemasking membrane comprising polymers that are insoluble in an acidic environment and soluble at pH 5 or higher as disclosed in U.S. Pat. No. 5,409,711 to Eurand International, SpA.

U.S. Application No. 20080113021 to Shen discloses dosage forms capable of being chewed or disintegrated in the oral cavity prior to swallowing that contain a plurality of particles that contain a propionic acid derivative, such as ibuprofen, and a taste-masking effective amount of a water soluble acid having a solubility greater than about 10 g/100 mL water at 20° C.; and a matrix that contains an acid having a solubility less than about 5 g/100 mL water at 20° C.

U.S. Pat. No. 6,117,452 To Fuisz Technologies Ltd. discloses microspheres that contain combinations of glyceryl monostearate and polyethylene glycol glyceryl palmitosterate. The reference disclosed that the microspheres can be readily treated, e.g., with taste-masking and/or controlled release coatings.

U.S. Pat. No. 5,405,617 to McNeil-PPC, Inc. discloses a method for preparing a pharmaceutical matrix without the use of organic and/or volatile solvents that includes melting a taste-masking amount of an aliphatic or fatty acid ester; admixing at least one pharmaceutical active with the molten aliphatic or fatty acid ester; and solidifying the admixture.

European Patent No. EP818992B1 to Eurand America, Inc. discloses a taste-masked, water-insoluble NSAID that contains individual microcapsules simultaneously microencapsulated with gelatin and cellulose acetate phthalate.

European Patent No. EP1301176B1 to Gattefosse Holding discloses a process for coating solid particles with a hot-melt agent.

European Patent Application No. EP2198856A1 to Reckitt Benckiser Healthcare discloses a process for preparing a granular composition of solidified melt granules comprising a NSAID drug as a continuous phase.

International Patent Application No. WO1994005260 to Affinity Biotech, Inc. discloses a method of masking the flavor of a drug that includes mixing the drug in particulate form into a lipid at a temperature below where significant drug degradation occurs and adding an emulsifier, a polymer and an aqueous dilution solution.

Despite the disclosures of the above patents and applications, a method for providing a tastemasked propionic acid derivative composition with reduced throat burn is still desired.

In accordance with an embodiment of the invention, propionic acid derivative particles are prepared as follows:
1. propionic acid derivative and wax are melted while mixing;
2. the molten propionic acid derivative/wax mixture is dispersed in hot water;
3. the hot dispersion is transferred into another container containing ambient/cold water;
4. the dispersed droplets of propionic acid derivative/wax congeal as a result of the rapid drop in temperature and form fine/spherical particles;
5. the fine/spherical particles are filtered and dried.

The process of the invention can be used to manufacture propionic acid derivative particles for use in pediatric and adult oral dosage forms. For example, the process of the invention can be used to manufacture taste masked particles for use in chewable, powder pack, suspension, confectionery and/or orally disintegrating dosage forms.

In one embodiment the particles of the current invention can be utilized in liquid dosage forms such as suspensions. In the embodiment wherein a suspension form is created utilizing the process of the current invention, the particles may or may not be dried prior to incorporation into the suspension vehicle. In one embodiment of the suspension, the suspension is created utilizing the process as follows:
1. propionic acid derivative and wax are melted while mixing;
2. the molten propionic acid derivative/wax mixture is dispersed in hot water or hot water containing pharmaceutically preferred suspending agents (ex. xanthan gum);
3. the hot dispersion is transferred into another container containing ambient/cold suspension vehicle;
4. the dispersed droplets of propionic acid derivative/wax congeal as a result of the rapid drop in temperature and form fine/spherical particles;
5. the suspension is completed by addition of the excipients, sweeteners, preservatives, and/or flavors;

According to another embodiment, the suspension is prepared by separating the congealed propionic acid/wax particles, drying and incorporating into a suspension by combining with excipients and water.

A preferred ratio of propionic acid derivative/wax for an immediate release dosage form is from about 80:20 to about 95:5. A more preferred ratio of propionic acid derivative/wax for an immediate release dosage form is 85:15.

The process of the invention can also be used to manufacture propionic acid derivative particles for use in sustained release dosage forms. Suitable sustained release dosage forms include compressed tablets, capsules, liquid filled capsules, bi-layer tablets, In one embodiment, the sustained release coated particles of the process of the current invention may be incorporated with immediate release particles of the proprionic acid derivative to create a dosage form with immediate release and sustained release characteristics. In another embodiment the particles of the current invention may be combined with additional active ingredient(s).

A preferred ratio of propionic acid derivative/wax for a sustained release dosage form is from less than about 80:more than about 20 to about 40:60. A more preferred ratio of propionic acid derivative/wax for a sustained release dosage form is from about 50:50 to about 70:30. A preferred ratio of propionic acid derivative/wax for a sustained release dosage form is 70:30. A preferred ratio of propionic acid derivative/wax for a sustained release dosage form is 50:50.

The process of the invention can be used to manufacture propionic acid derivative particles that range in size from about 50 microns to about 300 microns.

The process of the invention can be used to manufacture propionic acid derivative particles with a narrow particle size range.

According to the invention, a preferred propionic acid derivative is ibuprofen. Other proprionic acid derivatives for use in the process of the present invention include but are not limited to ketoprofen and dexibuprofen.

Other features and advantages of the present invention will be apparent from the detailed description of the invention and from the claims.

### BRIEF DESCRIPTION OF THE FIGURES

FIG. 1 is a graph showing the dissolution of ibuprofen tablets containing taste masked ibuprofen particles with 15% of glyceryl behenate and prepared in accordance with the invention.

FIG. 2 is a graph showing the dissolution profiles of sustained release ibuprofen particles with 30% and 50% of glyceryl behenate and prepared in accordance with the invention.

### DETAILED DESCRIPTION OF THE INVENTION

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified. In addition, all ranges set forth herein are meant to include any combinations of values between the two endpoints, inclusively.

As used herein, the term “immediate release” shall mean that the dissolution of the dosage form conforms to USP specifications for immediate release tablets containing the particular active ingredient employed. For example, for ibuprofen tablets, USP 35 specifies that in pH 7.2 phosphate buffer, using USP apparatus 2 (paddles) at 50 rpm, at least 80% of the ibuprofen contained in the dosage form is released within 60 minutes. See USP 35-NF 302012 Ibuprofen Tablets Monograph and General Chapter <711>.

Time release technology, also known as sustained-release, is a mechanism used in tablets or capsules to dissolve slowly and release a drug over time. The advantages of sustained-release tablets or capsules are that they can often be taken less frequently than immediate-release formulations of the same drug, and that they keep steadier levels of the drug in the bloodstream.

The term, “good mouth feel” shall mean the general sensory experience by the consumer during and after the oral consumption of the dosage form, including, but not limited, by chewable forms or and suspensions.

The term, “burn” is understood to mean the commonly identified peppery or irritating sensation in the throat and/or mouth, often noted when taking low melting propionic acid derivative compounds such as ibuprofen and related compounds. This burn is different than bitterness inasmuch as the addition of a sweetener is not effective in reducing the sensation. The burn can be expressed as a throat catch, or as a sudden cough reflex that results from the irritation.

Propionic acid derivatives are a well known class of analgesic compounds. As used herein propionic acid derivatives are understood to include, but are not limited to, ibuprofen, naproxen, benoxaprofen, naproxen sodium, flurbiprofen, fenoprofen, fenbuprofen, ketoprofen, indoprofen, pirprofen, carpofen, oxaprofen, pranoprofen, microprofen, tioxaprofen, suproprofen, alminoprofen, tiaprofenic acid, fluprofen and bucloxic acid. The structural formula is set forth in U.S. Pat. No. 4,923,898, which is hereby incorporated by reference. Propionic acid derivatives as defined herein are defined as pharmaceutically acceptable analgesics/non-steroidal anti-inflammatory drugs having a free —CH(CH₃)COOH or —CH₂CH₂COOH or a pharmaceutically acceptable salt group, such as —CH(CH₃)COO—Na+ or CH₂CH₂COO—Na+, which are typically attached directly or via a carbonyl functionality to an aromatic ring system.

Typical adult daily dosage of Over the Counter ibuprofen, a propionic acid derivative, is 200 mg to 1200 mg, with daily prescription dosage ranging up to 3200 mg/day.

Ibuprofen is a widely used, well known non-steroidal anti-inflammatory propionic acid derivative. Ibuprofen is chemically known as 2-(4-isobutylphenyl)-propionic acid. As used herein ibuprofen is understood to include 2-(4-isobutylphenyl)propionic acid as well as the pharmaceutically acceptable salts. Suitable ibuprofen salts include, for example, sodium, arginine, lysine, histidine, as well as other salts described in U.S. Pat. Nos. 4,279,926, 4,873,231, 5,424,075 and 5,510,385, the contents of which are incorporated by reference herein.

The formulation of the present invention may also contain pharmaceutically acceptable excipients, fillers, flavors, diluents, lubricants, disintegration agents, suspension agents, stabilizers, binders, colorants, carriers and the like. For example suitable carriers include lactose, starch, dicalcium phosphate, calcium sulfate, kaolin, mannitol and powdered sugar. Typical binders include starch gelatin, sugars (such as dextrose, mannitol, xylitol, sorbitol, maltodextrins, fructose, sucrose, molasses), and lactose, polyvinylpyrrolidone, polyethylene glycol, ethyl cellulose and waxes. Lubricants include boric acid, sodium benzoate, magnesium stearate, sodium acetate, sodium chloride, leucine, polyethylene glycol and the like. Typical disintegrants include, starch derived from wood, maize, potato, and rice, methylcellulose, magnesium silicates, aluminum silicates, sucrose, dextrose, maltodextrin, agar, alginic acid, wood products, guar gum, citric pulp, sodium lauryl sulfate and the like.

The present invention may be provided in liquid or semi-solid form, e.g., an elixir, suspension, syrup, gel, cream, ointment, or sugar cream confection such as a fondant or nougat. The liquid or semi-solid formulations are prepared using manufacturing methods and pharmaceutically acceptable surfactants, dispersants, sweeteners and diluents known in the art. Preferably the present invention is provided in tablets or other solid dosage forms and most preferably in a chewable form.

The invention will now be illustrated by, but is not intended to be limited to, the following example. In the example, it is understood that unless noted otherwise, all parts are weight percent.

### EXAMPLES

Specific embodiments of the present invention are illustrated by way of the following examples. This invention is not confined to the specific limitations set forth in these examples.

### Example 1

### Preparation of Melted Taste-Masked Particles Containing Ibuprofen with a Ratio of Drug: Glyceryl Behenate of 85:15

Approximately 85 g of ibuprofen USP and 15 g of glyceryl behenate, which is commercially available as Compritol ATO 888, from the Gattefosse corporation in Lyon, France, were added to a suitable vessel while mixing with a laboratory mixer at appropriate speed and heated to 80-90° C. until both ingredients melt. 200 g of purified water is added to a second suitable stainless steel vessel and heated to approximately 80-90° C. While mixing, the molten ibuprofen and glyceryl behenate mixture is added to the hot water. The dispersion of molten mixture of ibuprofen and glyceryl behenate and hot water is then added to a separate vessel containing 200 g of cold water (less than 10° C.) while mixing to congeal the ibuprofen/wax droplets. The resulting particles were filtered through a suitable stainless steel mesh screen, collected and dried at room temperature overnight in a desiccator. The resulting particles have a mean particle size range between 170 and 250 microns.

### Example 2

### Preparation of Chewable Tablet Comprising Taste-Masked Ibuprofen Particles from Example 1

The dried taste-masked ibuprofen particles from Example 1, and the materials in the table below were blended together in V-Blender and compressed using a rotary tablet press to a hardness of 4-7 kp.

TABLE 1
Formula of a Prototype Chewable Ibuprofen Tablet

| | Percent |
|---|---|
| Ingredients | (w/w) |
| Melted Taste-Masked Particles Containing Ibuprofen | 9.8 |
| (85% active) | |
| Dextrose Monohydrate | 83.2 |
| Crospovidone NF | 1.7 |
| Orange Flavor | 0.3 |
| Magnesium Stearate NF | 1.6 |
| Colloidal Silicon Dioxide NF | 0.1 |
| Fumaric Acid NF | 0.6 |
| Citric Acid USP | 0.3 |
| FD&C Yellow 6 Aluminum Lake | 0.2 |
| Acesulfame Potassium | 1.1 |
| Sucralose NF | 1.1 |
| TOTAL | 100.0 |

### Example 3

### Preparation of Taste-Masked Ibuprofen Suspension Utilizing Ratio of Ibuprofen: Glyceryl Behenate of 85:15

Utilizing the formula in Table 2, an in-situ taste-masked ibuprofen suspension was prepared. Ibuprofen and glyceryl behenate were melted in a 1500 mL glass beaker “A” at 80-90° C. In beaker “B”, citric acid and part xanthan gum were dissolved in about 300 mL purified water heated to 80-90° C. Contents of beaker B were added to the molten ibuprofen/wax combination in beaker A under continuous stirring. The temperature of beaker A was maintained at 80-90° C. The water in part II was at room temperature and placed in a third beaker “C” and cooled down to less than 10° C. Once the ibuprofen and the glyceryl behenate formed a uniform dispersion in water, the mixture was removed from the water bath and hotplate. The contents of beaker C were poured into beaker A and slowly and continually stirred at 1000-1500 RPM, as the molten ibuprofen and glyceryl behenate mixture congealed into fine particles. Xanthan gum (from Part III) was poured into glycerin and added to the mixture in beaker A. The remaining ingredients from part III were added into beaker A, and mixed for 5 minutes. The resultant suspension was stored in a suitable labeled container.

TABLE 2
Formula of a Prototype Ibuprofen Suspension

| Ingredients | Batch amt (g) |
|---|---|
| Part I | |
| Ibuprofen | 25.0 |
| Glyceryl Behenate | 4.4 |
| Citric Acid | 2.3 |
| Xanthan Gum | 1.0 |
| Purified Water | 300.0 |
| Part II | |
| Purified Water | 362.5 |
| Part III | |
| Acesulfame Potassium | 1.3 |
| Corn Starch | 18.8 |
| FD&C Red #40 | 0.1 |
| Cherry Flavors | 1.7 |
| Glycerin | 125.0 |
| Polysorbate 80 | 0.6 |
| Sodium Benzoate | 2.5 |
| Sucralose | 0.7 |
| Sucrose | 375.0 |
| Xanthan Gum | 1.3 |
| Total | 1222.0 |

### Example 4

### Preparation of Sustained Release Particles Containing Ibuprofen with a Ratio of Drug: Glyceryl Behenate of 70:30 and 50:50

### Part A: Ratio of Ibuprofen:Glyceyl Behenate of 70:30

Approximately 70 g of ibuprofen USP (70 μm grade) and 30 g of glyceryl behenate, which is commercially available as Compritol ATO 888, from the Gattefosse corporation in Lyon, France, were added to a suitable vessel while mixing with a laboratory mixer at approximately 50 RPM and heated to 80-90° C. 200 g of purified water is added to a second suitable stainless steel vessel and heated to approximately 80-90° C. while mixing. The ibuprofen and glyceryl behenate mixture is added to the hot water while mixing. The melted mixture of ibuprofen and glyceryl behenate and hot water are then added to a separate vessel containing 200 g of cold water (less than 10° C.) while mixing. The resulting particles were filtered through a 100 mesh stainless steel screen, collected and dried for 6 hours at 30° C. The resulting particles have a mean particle size range between 170 and 250 microns.

### Part B: Ratio of Ibuprofen:Glyceyl Behenate of 50:50

Approximately 50 g of ibuprofen USP (70 μm grade) and 50 g of glyceryl behenate, which is commercially available as Compritol ATO 888, from the Gattefosse corporation in Lyon, France, were added to a suitable vessel while mixing with a laboratory mixer at approximately 50 RPM and heated to 80-90° C. 200 g of purified water is added to a second suitable stainless steel vessel and heated to approximately 80-90° C. while mixing. The ibuprofen and glyceryl behenate mixture is added to the hot water while mixing. The melted mixture of ibuprofen and glyceryl behenate and hot water are then added to a separate vessel containing 200 g of cold water (less than 10° C.) while mixing. The resulting particles were filtered through a 100 mesh stainless steel screen, collected and dried for 6 hours at 30° C. The resulting particles have a mean particle size range between 170 and 250 microns.

### Example 5

### Preparation of Melted Taste-Masked Particles Containing Ibuprofen with a Ratio of Drug: Glyceryl Behenate of 85:15, Alternate Mixing Process

Approximately 85 g of ibuprofen USP (70 μm grade) and 15 g of glyceryl behenate, which is commercially available as Compritol ATO 888, from the Gattefosse corporation in Lyon, France, were added to a suitable vessel while mixing with a laboratory mixer at approximately 50 RPM and heated to 80-90° C. 200 g of purified water of water preheated to 80-90° C. is added to the mixture while mixing. 200 g of cold water (less than 10° C.) is then added to the same vessel while mixing. The resulting particles were filtered through a 100 mesh stainless steel screen, collected and dried for 6 hours at 30° C. The resulting particles have a mean particle size range between 170 and 250 microns.

### Example 6

### Dissolution of Particles

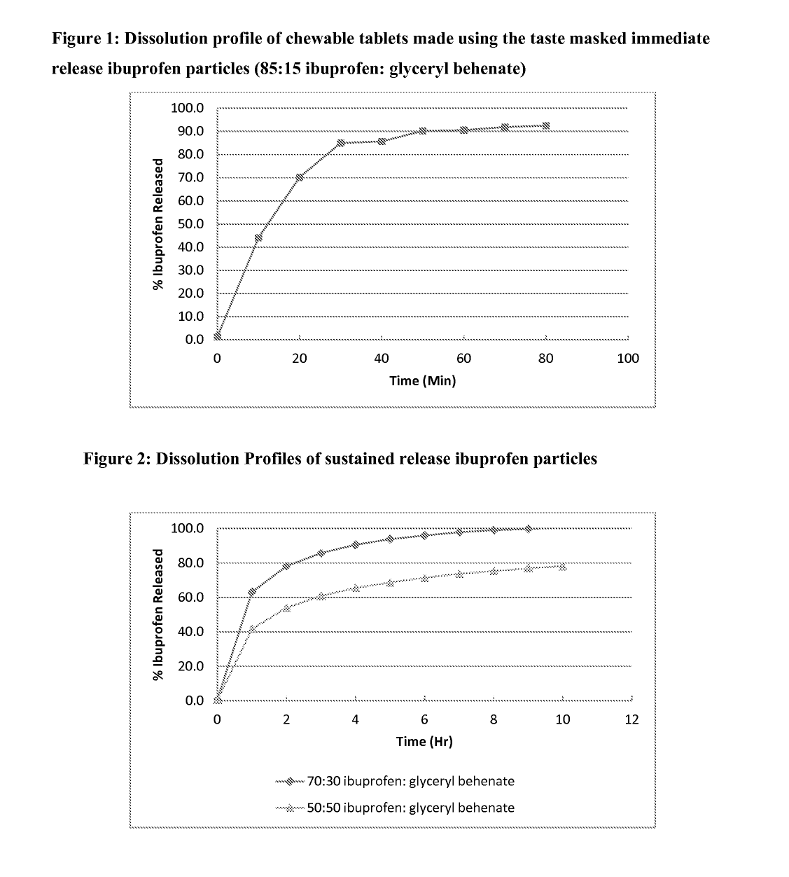

The chewable tablets from Example 2, containing the taste masked immediate release ibuprofen particles are tested for dissolution using USP Apparatus II. The dissolution medium was 900 mL of pH 7.2 phosphate buffer with paddle speed of 50 rpm. The dissolution data is presented in Table 3 and FIG. 1. Sustained release ibuprofen particles from example 4, part A (70:30 ibuprofen:glyceryl behenate) and example 4-part B (50:50 ibuprofen:glyceryl behenate) are also analyzed for dissolution using the same equipment over 10 hour period for ibuprofen content versus a standard prepared at 100% theoretical concentration. The dissolution data is shown in Table 4 and FIG. 2.

TABLE 3
Dissolution Analysis of Chewable tablets made
using the taste masked ibuprofen particles

70:30 ibuprofen:glyceryl behenate (n = 3)

| Time (Min) | Average | SD |
|---|---|---|
| 0 | 1.3 | 0.7 |
| 10 | 44.1 | 3.1 |
| 20 | 70.2 | 1.8 |
| 30 | 85.0 | 5.1 |
| 40 | 85.7 | 1.8 |
| 50 | 90.2 | 3.5 |
| 60 | 90.6 | 0.8 |
| 70 | 91.9 | 1.4 |
| 80 | 92.5 | 1.9 |

TABLE 4
Dissolution Analysis of sustained release ibuprofen particles

| | 70:30 ibuprofen:glyceryl | | 50:50 ibuprofen:glyceryl | |
|---|---|---|---|---|
| | behenate (n = 3) | | behenate (n = 3) | |
| Time (h) | Average | SD | Average | SD |
| 0 | 0.7 | 1.0 | 0.4 | 1.0 |
| 1 | 63.1 | 3.7 | 41.7 | 3.1 |
| 2 | 78.2 | 2.4 | 54.0 | 3.2 |
| 3 | 85.7 | 1.6 | 60.9 | 2.9 |
| 4 | 90.6 | 1.1 | 65.5 | 2.9 |
| 5 | 93.9 | 1.0 | 68.7 | 2.8 |
| 6 | 96.0 | 0.8 | 71.4 | 2.6 |
| 7 | 97.9 | 0.8 | 73.8 | 2.6 |
| 8 | 99.1 | 0.6 | 75.3 | 2.5 |
| 9 | 99.8 | 0.7 | 76.9 | 2.3 |
| 10 | 100.7 | 0.8 | 78.2 | 2.2 |

1. (canceled)

2. A method for preparing a suspension pharmaceutical formulation comprising:
melting a propionic acid derivative and a wax while mixing;
adding hot water containing pharmaceutical excipients to the molten propionic acid derivative/wax mixture; and
adding cold water, wherein the dispersed droplets of propionic acid derivative/wax congeal and form particles.
